(19) United States
(12) Patent Application Publication
(10) Pub. No.: US 2011/0190497 A1
(43) Pub. Date: Aug. 4, 2011

(54) Process for production of 2,5 dioxopyrrolidine 3 carboxylate

(76) Inventors: Daisuke Tanaka, Osaka-shi (JP); Hiroaki Tanaka, Osaka-shi (JP)

(21) Appl. No.: 12/310,996

(22) PCT Filed: Sep. 26, 2007
(86) PCT No.: PCT/JP2007/068631

§ 371 Date: Mar. 16, 2009

(30) Foreign Application Priority Data
Sep. 29, 2006 (JP) 2006-268314

(51) Int. Cl. C07D 487/20; C07D 207/416; C07D 405/04

(52) U.S. Cl. 544/231; 548/531; 548/518

(57) ABSTRACT

The present invention provides a novel intermediate which enable to prepare tetrahydropyrrolo[1,2-a]pyrazin-4-spiro-3′-pyrrolidine derivatives such as Ranirestat being promising therapeutic agents for diabetic complications in a short process and in an economically advantageous and safe manner, and a process for preparing the same. That is, the present invention provides a process for preparing a compound of the following formula (I) wherein R¹ is an amino group protected with a protecting group, etc., and R² is a lower alkyl group, etc., comprising the following steps (1) and (2): (1) a step of converting a cyano group in a compound of the following formula (II) wherein n and m are each independently 0 or 1; provided when n is 0 and m is 1, then R² and R³ are the same or different protecting groups for a carboxyl group; and when n is 1 and m is 0, then R² and R³ are the same protecting groups for a carboxyl group; and R¹ is as defined above, into a carbamoyl group in the presence of divalent palladium compound(s), primary amide(s) and water; and (2) a step of cyclizing the product obtained in the step (1).

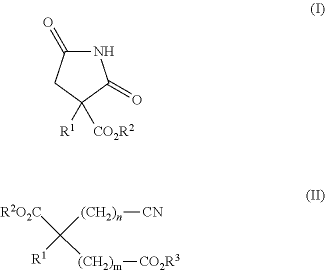

### TECHNICAL FIELD

This invention relates to a process for preparing 2,5-dioxopyrrolidine-3-carboxylates useful as a key intermediate of tetrahydropyrrolo[1,2-a]pyrazin-4-spiro-3′-pyrrolidine derivatives being useful as a therapeutic agent for diabetic complications.

### BACKGROUND ART

Tetrahydropyrrolo[1,2-a]pyrazin-4-spiro-3′-pyrrolidine derivatives which are promising therapeutic agents for diabetic complications showing a potent aldose reductase inhibitory activity are disclosed in the literature (for example, see JP-A-5-186472; and J. Med. Chem., 1998, 41, p.4118 to 4129). Also Ranirestat [AS-3201; (3R)-2′-(4-bromo-2-fluorobenzyl)spiro[pyrrolidin-3,4′(1′H)-pyrrolo[1,2-a]pyra zine]-1′,2,3′,5(2H1-tetraone] selected among these derivatives has been developed clinically. 2,5-Dioxopyrrolidine-3-carboxylates are key intermediates of these derivatives, and some processes for preparing the same are disclosed in the literatures (for example, JP-A-5-186472, JP-A-6-192222 and J. Med. Chem., 1998, 41, p. 4118 to 4129). Among them, a process shown in following Scheme is useful as an industrial process.

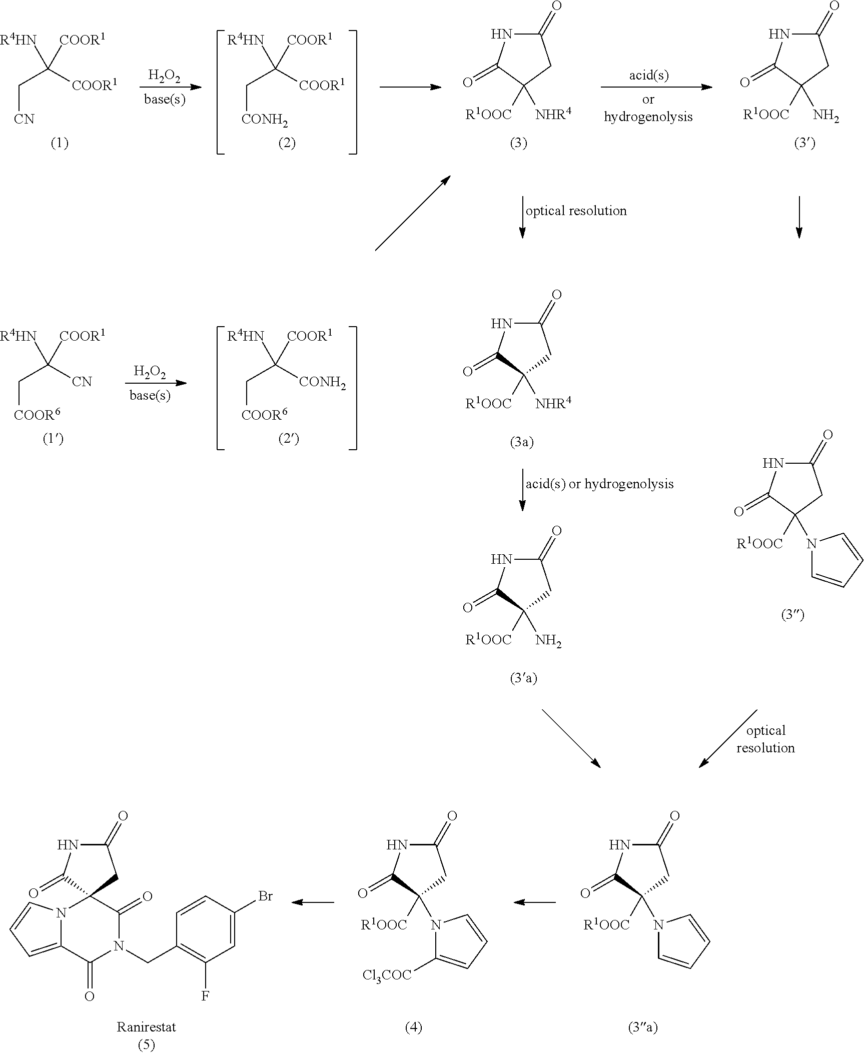

wherein R¹ is a protecting group for a carboxyl group, R⁴ is a group cleavable by hydrogenolysis or a tert-butoxycarbonyl group.

In the above process, in order to obtain a desirable intermediate 2,5-dioxopyrrolidine-3-carboxylates (3), (3′) and (3″), hydrogen peroxide is used as a reagent for converting a cyano group in a compound (1) and (1′) into an amide group, thus resulting in producing a compound (2) and (2′). There is a difficulty in controlling the reaction since this step using hydrogen peroxide is an exothermic reaction and thus often happens to foam violently. Thus it is desirous of a process for preparing 2,5-dioxopyrrolidine-3-carboxylates with easy control of the reaction and in safer manner. On the other hand, several processes of converting a cyano group into an amide group are known, but it is necessary to avoid a step requiring for a strong acidic condition or high reaction temperature since a chemical structure of 2,5-dioxopyrrolidine-3-carboxylates is easily subjected to a hydrolysis.

### DISCLOSURE OF INVENTION

### Problems to be Solved by Invention

An object of the present invention is to provide a industrially-advantageous process for preparing 2,5-dioxopyrrolidine-3-carboxylates useful as a synthetic intermediate of tetrahydropyrrolo[1,2-a]pyrazin-4-spiro-3′-pyrrolidine derivatives which are promising therapeutic agents for diabetic complications showing a potent aldose reductase inhibitory activity without use of hydrogen peroxide and in safe manner and efficiently.

### Means for Solving Problem

The present inventors have intensively studied in order to achieve the above-mentioned objects, specifically on a process for converting a cyano group into an amide group using an easily available metallic compound catalyst under mild conditions, and a process for preparing 2,5-dioxopyrrolidine-3-carboxylates comprising the preceding process and have found that a process using a particular metallic compound catalyst is useful as a process for converting a cyano group into an amide group which relates to the problems to be solved by invention and also the process is applicable as a one-pot process for preparing 2,5-dioxopyrrolidine-3-carboxylates efficiently and have accomplished the present invention. That is, the present invention provides a novel process for preparing 2,5-dioxopyrrolidine-3-carboxylates and relates to the following embodiments:

[1] A process for preparing a compound of the following formula (I):

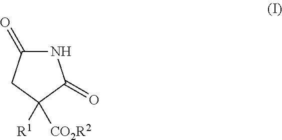

wherein R¹ is an amino group protected with a protecting group, a hydrazino group protected with a protecting group or a pyrrol-1-yl group; and R² is a lower alkyl group, a cycloalkyl group, a cycloalkyl-lower alkyl group, an optionally substituted aryl group, or an optionally substituted aryl-lower alkyl group,
which comprises the following steps (1) and (2):

(1) a step of converting a cyano group in a compound of the following formula (II):

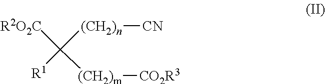

wherein n and m are each independently 0 or 1;
provided when n is 0 and m is 1, then R² and R³ are the same or different protecting groups for a carboxyl group; and when n is 1 and m is 0, then R² and R³ are the same protecting groups for a carboxyl group; and

R¹ is as defined above,

into a carbamoyl group in the presence of divalent palladium compound(s), primary amide(s) and water; and

(2) a step of cyclizing the product obtained in the step (1).

[2] The process as set forth in [1] wherein the divalent palladium compound is palladium(II) chloride, palladium(II) acetate or palladium(II) trifluoroacetate and the primary amide is acetamide, propionamide, n-butylamide or isobutylamide.

[3] The process as set forth in [1] wherein the step (1) is a step of converting a cyano group in the compound of the formula (II) as set forth in

[2] into a carbamoyl group in the presence of divalent palladium compound(s), primary amide(s), water and organic solvent(s) without a cyano group.

[4] The process as set forth in [3] wherein the divalent palladium compound is palladium(II) chloride, palladium(II) acetate or palladium(II) trifluoroacetate, the primary amide is acetamide, propionamide, n-butylamide or isobutylamide, and the organic solvent without a cyano group is a single solvent selected from the group consisting of tetrahydrofuran, methanol, ethanol, isopropanol, tert-butanol, ethyl acetate, N,N-dimethylformamide and dimethyl sulfoxide or a mixed solvent of a combination of two or three kinds thereof.

[5] The process as set forth in any one of [1] to [4] wherein the step (2) is a step of cyclizing the product obtained in the step (1) with base(s).

[6] The process as set forth in [5] wherein the step (2) is performed in the presence of chelating reagent(s).

[7] The process as set forth in any one of [1] to [5] wherein the step (1) comprises a step of removing the divalent palladium compound(s) from the resulting reaction mixture of the step (1).

[8] The process as set forth in [7] wherein the step of removing the divalent palladium compound(s) from the resulting reaction mixture of the step (1) is a step of washing the resulting reaction mixture of the step (1) with aqueous inorganic acid solution(s).

[9] The process as set forth in any one of [1] to [8] for preparing the compound of the formula (I) wherein R¹ is an amino group protected with a protecting group cleavable by hydrogenolysis, a hydrazino group protected with a protecting group cleavable by hydrogenolysis or a pyrrol-1-yl group, and R² is a lower alkyl group,

wherein the protecting group for a carboxyl group in the compound of the formula (II) is a lower alkyl group.

[10] A process for preparing Ranirestat comprising a step of preparing the compound of the formula (I) from the compound of the formula (II) as set forth in any one of [1] to [9], and a step of converting the compound of the formula (I) into Ranirestat.

An improved process for preparing an intermediate 2,5-dioxopyrrolidine-3-carboxylates according to the invention can be used to efficiently prepare Ranirestat useful as a medicament.

That is, the invention also provides an improved process for preparing Ranirestat. For example, when R¹ group is an amino group protected with a protecting group or a hydrazino group protected with a protecting group in the formula (II), the process for preparing Ranirestat includes the following steps:

(i) a step of converting a cyano group in the above compound of the formula
(II) wherein R¹ is an amino group protected with a protecting group or a hydrazino group protected with a protecting group into a carbamoyl group;
(ii) a step of cyclizing the above product of the step (i) to prepare a compound of a formula (I) wherein R¹ is an amino group protected with a protecting group or a hydrazino group protected with a protecting group;
(iii) a step of deprotecting the above product of the step (ii) by hydrogenolysis or strong acid(s);
(iv) a step of performing an optical resolution on the above product of the step (iii) to produce an optically active substance (R isomer);
(v) a step of converting an amino group of the above product of the step (iv) into a pyrrol-1-yl group;
(vi) a step of converting a pyrrol-1-yl group of the above product of the step
(v) into a 2-trichloroacetylpyrrol-1-yl group; and
(vii) a step of reacting the above product of the step (vi) with 4-bromo-2-fluorobenzylamine to convert it into Ranirestat.

As an alternative method, in the above process wherein R¹ group in the compound of the formula (II) of the step (i) is an amino group protected with a protecting group, the order of the step (iii) and the step (iv) are also interchangeable in the above process to prepare Ranirestat.

As a further alternative method, when R¹ is a pyrrol-1-yl group in the formula (II), the process for preparing Ranirestat comprising the following steps can also be provided:

(a) a step of converting a cyano group in the above compound of the formula (II) into a carbamoyl group;
(b) a step of cyclizing the above product of the step (a) to prepare the compound of the formula (I) wherein R¹ is a pyrrol- 1-yl group;
(c) a step of performing an optical resolution on the above product of the step (b) to produce an optically active substance (R isomer);
(d) a step of converting a pyrrol- 1-yl group in the above product of the step (c) into a 2-trichloroacetylpyrrol-1-yl group; and
(e) a step of reacting the above product of the step (d) with 4-bromo-2-fluorobenzylamine to convert it into Ranirestat.

### Effect of the Invention

The process of the present invention is a process for preparing 2,5-dioxopyrrolidine-3-carboxylates useful as an intermediate for Ranirestat without use of a dangerous reagent such as hydrogen peroxide under mild reaction conditions, which further can be expected to increase a yield of the compound, which thus is useful as an industrial process of the same.

### Best Modes for Carrying out the Invention

The present invention is explained in more detail below.

The following are definitions of terms used in this description and claims. The initial definition provided for a group or term herein applies to that group or term throughout the description and claims, individually or as part of another group, unless otherwise indicated.

The “amino group protected with a protecting group” is an amino group protected with a protecting group used routinely in a peptide synthesis art, and specifically includes an amino group protected with a protecting group cleavable by hydrogenolysis or strong acid(s), etc. The preferred protecting group includes a protecting group cleavable by hydrogenolysis, such as a benzyloxycarbony group in which the benzene ring moiety may be optionally substituted by one to three atom(s) or group(s) independently selected from the group consisting of halogen atom, lower alkyl group, lower alkoxy group and nitro group, etc. Specific examples of the protecting group cleavable by hydrogenolysis include benzyloxycarbony group, 4-chlorobenzyloxycarbony group, 4-methylbenzyloxycarbony group, 2-methoxybenzyloxycarbony group and 4-nitrobenzyloxycarbony group, etc. Specific examples of a protecting group cleavable by strong acid(s) include tert-butoxycarbonyl group, etc. Preferred specific examples of the amino group protected with a protecting group include benzyloxycarbonylamino group, 4-chlorobenzyloxycarbonylamino group, 4-methylbenzyloxycarbonylamino group, 2-methoxybenzyloxycarbonylamino group and 4-nitrobenzyloxycarbonylamino group, etc.

The “hydrazino group protected with a protecting group” is a hydrazino group protected with a protecting group used routinely in a peptide synthesis art, and specifically includes a hydrazino group protected with a protecting group cleavable by hydrogenolysis or strong acid(s), etc. The preferred protecting group is a protecting group cleavable by hydrogenolysis. Specific examples of the protecting group cleavable by hydrogenolysis or strong acid(s), etc., are the same described as above in a definition of the amino group protected with a protecting group. The preferred examples of the hydrazino group protected with a protecting group include N,N′-bis(benzyloxycarbonyl)hydrazino group, N,N′-bis(4-chlorobenzyloxycarbonyl)hydrazino group, N,N′-bis(4-methylbenzyloxycarbonyl)hydrazino group, N,N′-bis(2-methoxybenzyloxycarbonyl)hydrazino group and N,N′-bis(4-nitrobenzyloxycarbonyl)hydrazino group, etc.

The “lower alkyl group” is a straight chain or branched chain alkyl group having 1 to 6 carbon atom(s) (C₁₋₆ alkyl group), specifically such as methyl group, ethyl group, propyl group, isopropyl group, butyl group, isobutyl group, tert-butyl group, pentyl group, isopentyl group, neopentyl group and hexyl group, etc.

The “cycloalkyl group” is a cyclic alkyl group having 3 to 8 carbon atoms (C₃₋₈ cycloalkyl group), specifically such as cyclopropyl group, cyclobutyl group, cyclopentyl group, cyclohexyl group, cycloheptyl group and cyclooctyl group.

The “cycloalkyl-lower alkyl group” is a lower alkyl group substituted by cycloalkyl group, and preferred specific examples include cyclopropylmethyl group, cyclopentylmethyl group and cyclohexylmethyl group.

The “optionally substituted aryl group” is an aryl group optionally substituted by one to three atom(s) or group(s) selected from the group consisting of halogen atom, lower alkyl group, lower alkoxy group and nitro group (the aryl group is referred herein as a phenyl group and a fused polycyclic aromatic hydrocarbon group containing benzene ring), and preferred specific examples include phenyl group, naphthyl group, 4-chlorophenyl group, 4-methylphenyl group and 2-methoxyphenyl group, etc.

The “optionally substituted aryl-lower alkyl group” is a lower alkyl group substituted by an optionally substituted aryl group, and preferred specific examples include benzyl group, 4-chlorobenzyl group, 4-methylbenzyl group, 4-methoxybenzyl group and 2-methoxybenzyl group, etc.

Specific examples of “divalent palladium compound(s)” include palladium(II) chloride, palladium(II) acetate and palladium(II) trifluoroacetate, etc.

The “primary amide(s)” is an organic compound containing a carbamoyl group, preferably a straight chain or branched chain saturated hydrocarbon having 1 to 6 carbon(s) containing a carbamoyl group. Preferred specific examples include acetamide, propionamide, n-butylamide, isobutylamide, etc., among them most preferably acetamide.

The “protecting group(s) for a carboxyl group” is a protecting group for a carboxyl group which is used routinely in a peptide synthesis art and cannot be deprotected simultaneously with performance of a deprotection of an amino group protected with a protecting group or a hydrazino group protected with a protecting group. Preferred protecting group for a carboxyl group includes a lower alkyl group or an optionally substituted aryl group, among them preferably a lower alkyl group.

The “chelating reagent(s)” is a compound being capable of coordinating to palladium. Specific examples include an organic base such as N,N,N′,N′-tetramethylethylenediamine (hereinafter, abbreviated as “TMEDA”), triethylamine, dibutylamine, 1,10-phenanthroline, and an organic phosphorus compound such as triphenylphosphine, etc.

The process of the invention is explained as follows:

2,5-Dioxopyrrolidine-3-carboxylates of a formula (I) can be prepared by the following method:

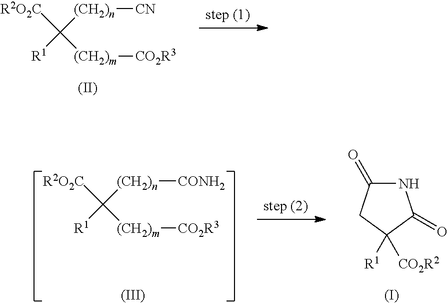

wherein R¹, R², R³, n and m are described as above.

The step (1) is a step of hydrating a cyano group in the compound of the formula (I) by reacting it with primary amide(s) in the presence of divalent palladium compound(s) in water and appropriate organic solvent(s) to prepare the compound of the formula (III). This step can be performed in the same manner to that described in Org. Lett., 2005, 7, p.5237-5239. The amount of the divalent palladium compound(s) is not limited otherwise, but preferably is a catalytic amount to that of the compound of the formula (II) such as 0.001 to 0.5 equivalents. The amount of the primary amide(s) is usually an amount of 1 to 50 equivalents to that of the compound of the formula (II). The amount of water is usually 1 to 50 mL per 1 g of the compound of the formula (II).

The organic solvent(s) used in the step (1) is organic solvent(s) without a cyano group, preferably organic solvent(s) without a cyano group miscible with water. The organic solvent(s) includes for example, alcohol solvents such as methanol, ethanol, isopropanol, tert-butanol; ester solvents such as ethyl acetate; ether solvents such as tetrahydrofuran; polar aprotic solvents such as N,N-dimethylformamide, dimethyl sulfoxide, etc., among them preferably tetrahydrofuran. These organic solvents can be used alone or in a combination of two or more kinds thereof. The amount used of the organic solvent(s) is usually 0.5 to 2 mL per 1 mL of water. The reaction temperature is not limited otherwise, but preferably is a room temperature (about 5° C. to about 35° C.).

After a completion of the reaction of the step (1), removal of the divalent palladium compound(s) from the reaction mixture can also increase a yield and a purity of the compound of the formula (1) in the step (2). The method for removing the divalent palladium compound(s) includes a method of washing the reaction mixture produced in the step (1) with aqueous inorganic acid solution(s). Specific examples of the aqueous inorganic acid solution(s) include aqueous hydrochloric acid solution, aqueous sulfuric acid solution and aqueous phosphoric acid solution, etc., among them preferably aqueous hydrochloric acid solution. The concentration of the aqueous inorganic acid solution(s) is usually 0.1 to 2 M.

A step of (2) is a step of reacting a carbamoyl group in the compound of the formula (III) with an ester to prepare the compound of the formula (1). The ring-closure reaction of the step (2) can proceed with consecutively under the reaction condition of the step (1) without isolating the compound of the formula (III) and can also be performed continuously in the same reactor.

On the other hand, an addition of base(s) after a completion of the reaction of the step (1) can also reduce a time spent in the ring-closure reaction since a reaction rate of this ring-closure reaction is usually slow. Specific examples of the base(s) include inorganic base(s) such as potassium carbonate, sodium carbonate or sodium bicarbonate, and organic base(s) such as triethylamine, pyridine, 1,8-diazabicyclo[5.4.0]undeca-7-ene, sodium ethoxide and potassium tert-butoxide, etc. Preferred base(s) is inorganic base(s) such as potassium carbonate, sodium carbonate or sodium bicarbonate, etc. The amount used of the base(s) is selected from a catalytic amount to an excess amount to that of the compound of the general formula (III), preferably 1 to 5 equivalent(s) to that of the compound of the formula (II). The reaction temperature is not limited otherwise, but is preferably a room temperature. The solvent(s) include methanol, ethanol, isopropanol, tetrahydrofuran, acetonitrile and water, etc., which can be used alone or in a combination of two or more kinds thereof.

An addition of chelating reagent(s) after a completion of the reaction of the step (1) can also be increased a yield and a purity of the compound of the formula (I) in the step (2). The amount of the chelating reagent(s) is usually 0.5 to 10 equivalents to that of the compound of the formula (II).

The compound of the formula (II) can be prepared according to a method described in the patent documents 1 and 2 and the non-patent document 1 as aforementioned or a similar method thereto.

The patent documents 1 and 2 and the non-patent document 1 describe a process for preparing Ranirestat using the compound of the formula (I) (with the proviso that R¹ in the compound is a group other than hydrazino group protected with a protecting group). Thus the process of the present invention can be applied in the process for preparing Ranirestat.

### EXAMPLES

The present invention is illustrated in more detail below by Examples, but the present invention should not be construed to be limited thereto. The compounds were characterized by proton nuclear magnetic resonance spectrum (¹H NMR), carbon 13 nuclear magnetic resonance spectrum (¹³C NMR), and mass spectrum (MS) analyses. Tetramethyl silane is used as an internal standard in the nuclear magnetic resonance spectrum analyses. For the abbreviations in Examples, Et is ethyl group and Cbz is benzyloxycarbonyl group.

### Example 1

Preparation of Ethyl 3-benzyloxycarbonylamino-2,5-dioxopyrrolidine-3-carboxylate:

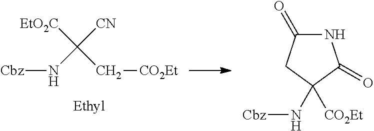

3-benzyloxycarbonylamino-3-ethoxycarbonyl-3-cyanopropionate (1.0 g) and acetamide (1.7 g) were dissolved in 50% (v/v) tetrahydrofuran-water solution (30 mL) and thereto was added palladium(II) chloride (64 mg). This mixture was stirred at room temperature for 15 hours. The reaction mixture was extracted with ethyl acetate and the extract was washed with 0.5 M hydrochloric acid three times and water once. This ethyl acetate solution was dried over magnesium sulfate, filtered and then the filtrate was concentrated. The resulting residue was dissolved in the 50% (v/v) tetrahydrofuran-water solution (30 mL) and thereto was added sodium carbonate (0.46 g) and the mixture was stirred at room temperature for 5 hours. The reaction mixture was adjusted to pH 1 with 0.5 M hydrochloric acid and was extracted with ethyl acetate. This ethyl acetate solution was washed with water, dried over magnesium sulfate and filtered. The filtrate was concentrated to give a desired product as crystal (0.93 g, 100%).
¹H NMR (400 MHz, CDCl₃, 22° C.) δ: 8.77 (1H, br), 7.39-7.33 (5H, m), 6.29 (1H, br), 5.15 (1H, d, J=12.0 Hz), 5.08 (1H, d, J =12.4 Hz), 4.32 (2H, q, J=7.1 Hz), 3.22 (1H, d, J=18.0 Hz), 3.14 (1H, d, J =18.0 Hz), 1.29 (3H, t, J=7.0 Hz). ¹³C NMR (100 MHz, CDCl₃, 23° C.) δ: 173.2, 172.1, 166.5, 154.9, 128.6, 128.5, 128.2, 67.7, 64.5, 64.1, 40.8, 13.8.

### Example 2

Preparation of Ethyl 3-benzyloxycarbonylamino-2,5-dioxopyrrolidine-3-carboxylate:

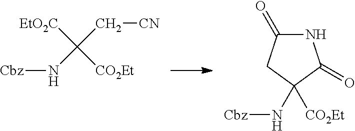

Diethyl 2-benzyloxycarbonylamino-2-cyanomethylmalonate (348 mg) and acetamide (591 mg) were dissolved in 50% (v/v) tetrahydrofuran-water solution (6 mL) and thereto was added palladium(II) chloride (17.7 mg) and the mixture was stirred at room temperature overnight. The reaction mixture was extracted with ethyl acetate and the extract was washed with 1 M hydrochloric acid three times and waster once. This ethyl acetate solution was dried over magnesium sulfate, filtered and the filtrate was concentrated. The resulting residue was dissolved in 50% (v/v) tetrahydrofuran-water solution (6 mL) and thereto was added sodium carbonate (0.46 g) and the mixture was stirred at room temperature for 1 hour. The reaction mixture was adjusted to be acidic with 1 M hydrochloric acid and extracted with ethyl acetate. This ethyl acetate solution was washed with water, dried over magnesium sulfate and filtered. The filtrate was concentrated to give the desired product (310 mg, 97%) as amorphous.

### Example 3

Preparation of Ethyl 3-benzyloxycarbonylamino-2,5-dioxopyrrolidine-3-carboxylate:

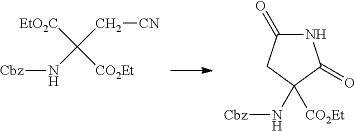

Diethyl 2-benzyloxycarbonylamino-2-cyanomethylmalonate (348 mg) and acetamide (591 mg) were dissolved in 50% (v/v) tetrahydrofuran-water solution (6 mL) and thereto was added palladium(II) chloride (17.7 mg) and the mixture was stirred at room temperature overnight. To this mixture was added TMEDA (0.038 mL) and after stirring for 5 minutes, thereto was added sodium carbonate (159 mg) and the mixture was stirred at room temperature for 1 hour. This reaction mixture was adjusted to be acidic with 1 M hydrochloric acid and extracted with ethyl acetate three times. This ethyl acetate solution was washed with water and saturated brine, dried over magnesium sulfate and filtered. The filtrate was concentrated to give a residue, which was purified by a column chromatography on silica gel (hexane: ethyl acetate=2:1) to give the desired product (294 mg, 92%) as oil.

### Example 4

Preparation of Ethyl

3-benzyloxycarbonylamino-2,5-dioxopyrrolidine-3-carboxylate:

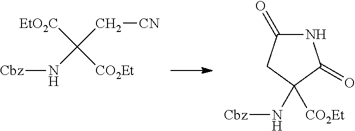

Diethyl 2-benzyloxycarbonylamino-2-cyanomethylmalonate (348 mg) and acetamide (591 mg) were dissolved in 50% (v/v) tetrahydrofuran-water solution (6 mL) and thereto was added palladium(II) chloride (17.7 mg) and the mixture was stirred at room temperature overnight. To this reaction mixture was added sodium carbonate (159 mg) and the mixture was stirred at room temperature for 50 minutes. This reaction mixture was adjusted to be acidic with 1 M hydrochloric acid and was extracted with ethyl acetate three times. This ethyl acetate solution was washed with water and saturated brine, dried over magnesium sulfate and filtered. The filtrate was concentrated to give a residue, which was purified by column chromatography on silica gel to give the desired product (293 mg, 92%) as oil.

### Example 5

Preparation of ethyl 3-(1-pyrrolyl)-2,5-dioxopyrrolidine-3-carboxylate:

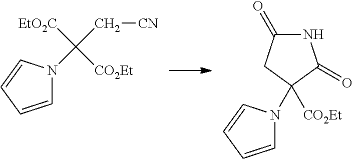

Diethyl 2-(1-pyrrolyl)-2-cyanomethylmalonate (264 mg) and acetamide (591 mg) were dissolved in 50% (v/v) tetrahydrofuran-water solution (6 mL), and thereto was added palladium(II) chloride (17.7 mg) and the mixture was stirred at room temperature for 3 days. Thereto was added more palladium(II) chloride (24.0 mg) and the mixture was stirred at room temperature for 2 days. To this reaction mixture was then added sodium carbonate (159 mg) and the mixture was stirred at room temperature for 45 minutes. This reaction mixture was extracted with ethyl acetate and the extract was washed with 1 M hydrochloric acid three times and water once. This ethyl acetate solution was dried over magnesium sulfate and filtered. The filtrates was concentrated to give a residue, which was purified by column chromatography on silica gel (n-hexane: ethyl acetate=2:1) to give a desired product (151 mg, 54%) as amorphous. ¹H NMR (400 MHz, CDCl₃, 23° C.) δ: 9.05 (1H, br), 6.94 (1H, t, J=2.2 Hz), 6.26 (1H, t, J=2.2 Hz), 4.28 (2H, q, J=7.2 Hz), 3.59 (1H, d, J=17.6 Hz), 3.36 (1H, d, J=18.0 Hz), 1.26 (3H, t, J=7.2 Hz). ¹³C NMR (100 MHz, CDCl₃, 24° C.) δ: 172.7, 170.5, 166.8, 120.0, 110.1, 68.6, 63.9, 41.9, 13.8. MS (APCI): 237(M+H).

### Reference Example 1

Preparation of 2-[N,N′-bis(benzyloxycarbonyl)hydrazino]-2-ethoxycarbonylmethyl-2-ethyl cyanoacetate:

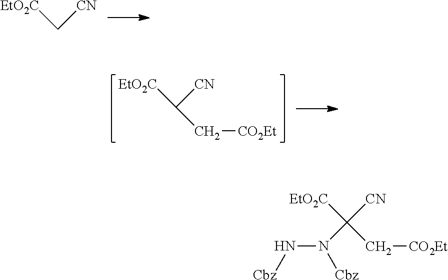

(1) To a solution of ethyl cyanoacetate (1.1 mL) in ethanol (10 ml) was added sodium ethoxide (20% ethanol solution, 3.4 g) slowly under ice-cooling, and then the mixture was stirred for 5 minutes. To this mixture was added ethyl bromoacetate (1.3 mL) and the mixture was stirred at room temperature for 1 hour. The reaction mixture was diluted with diisopropyl ether, washed with water, dried (MgSO₄), filtered and then the filtrate was concentrated to give an oil. This was purified by flash column chromatography (n-hexane: ethyl acetate=10:1 to 8:1) to give a reaction product (1.59 g).

(2) The above reaction product was dissolved in ethyl acetate (20 mL) and thereto were added dibenzyl azocarboxylate (895 mg), then potassium carbonate (41.5 mg) at room temperature. This reaction solution was stirred at room temperature for 15 minutes, filtered through a Celite pad and the filtrate was concentrated to give oil. This was purified by column chromatography on silica gel (n-hexane: ethyl acetate=3:1 to 2:1) to give the desired product (1.15 g, 23%) as crystal. MS (APCI): 498(M+H).

### Example 6

Preparation of Ethyl 3-[N,N′-bis(benzyloxycarbonyl)hydrazino]-2,5-dioxopyrrolidine-3-carboxylate:

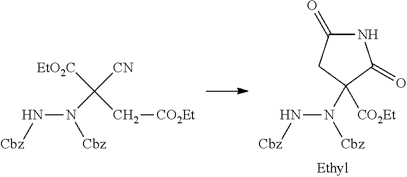

2-[N,N′-bis(benzyloxycarbonyl)hydrazino]-2-ethoxycarbonylmethyl-2-cyano acetate (301 mg) and acetamide (357 mg) were suspended in 50% (v/v) tetrahydrofuran-water solution (10 mL), subsequently thereto was added palladium(II) chloride (10.7 mg). This mixture was stirred at room temperature overnight. To this reaction mixture was added sodium carbonate (96 mg) and the mixture was stirred for 15 minutes, and then this reaction solution was adjusted to be acidic with 1 M hydrochloric acid and extracted with ethyl acetate three times. This ethyl acetate solution was washed with water, followed by saturated brine, and dried over magnesium sulfate, filtered and the filtrate was concentrated. The resulting residue was purified by column chromatography on silica gel (n-hexane: ethyl acetate=2: 1) to give the desired product (152 mg, 54%) as oil.

¹H NMR (300 MHz, DMSO-d₆, 120° C.) δ: 11.4 (1H, br), 9.66 (1H, br), 7.35-7.25 (10H, m), 5.15-5.02 (4H, m), 4.14 (2H, q, J=7.1 Hz), 3.40 (1H, d, J=18.3 Hz), 3.17 (1H, d, J=18.2 Hz), 1.14 (3H, t, J=7.1 Hz).

### Example 7

Preparation of ethyl 3-[N,N′-bis(benzyloxycarbonyl)hydrazino]-2,5-dioxopyrrolidine-3-carboxylate:

[symbol]

Ethyl

2-[N,N′-bis(benzyloxycarbonyllhydrazino]-2-ethoxycarbonylmethyl-2-cyano acetate (301 mg) and acetamide (358 mg) were suspended in 50% (v/v) tetrahydrofuran-water solution (10 mL), and subsequently thereto was added palladium(II) chloride (12.5 mg). This mixture was stirred at room temperature overnight. This reaction mixture was diluted with ethyl acetate and washed with 1 M hydrochloric acid four times, water twice and saturated brine once successively, dried over magnesium sulfate, filtered and the filtrate was concentrated. The resulting residue was suspended in a mixed solution of tetrahydrofuran-water (1:1 v/v, 10 mL) and thereto was added sodium carbonate (97.6 mg) and the mixture was stirred at the same temperature for 3 hours. This reaction mixture was adjusted to be acidic with 1 M hydrochloric acid and extracted with ethyl acetate three times. This ethyl acetate solution was washed with water, followed by saturated brine and dried over magnesium sulfate, filtered and the filtrate was concentrated. The resulting residue was purified by column chromatography on silica gel (n-hexane: ethyl acetate=2:1) to give the desire product (254mg 89%) as oil.

### Example 8

Preparation of Ethyl

3-[N,N′-bis(benzyloxycarbonyl) hydrazino]-2.5-dioxopyrrolidine-3-carboxylate:

[symbol]

Ethyl

2-[N,N′-bis(benzyloxycarbonyl)hydrazino]-2-ethoxycarbonylimethyl-2-cyano acetate (302 mg) and acetamide (362 mg) were suspended in 50% (v/v) tetrahydrofuran-water solution (10 mL), and subsequently thereto was added palladium(II) chloride (13.0 mg). This reaction mixture was stirred at room temperature overnight. To this reaction mixture were added TMEDA (28.0 μp, then sodium carbonate (97.0 mg) and the mixture was stirred at the same temperature for 3 hours. This reaction mixture was adjusted to be acidic with 1 M hydrochloric acid and extracted with ethyl acetate three times. This ethyl acetate solution was washed with water, followed by saturated brine, and dried over magnesium sulfate, filtered and the filtrate was concentrated. The resulting residue was purified by flash column chromatography (n-hexane: ethyl acetate=2:1) to give the desired product (241 mg, 85%) as oil.

### Example 9

Preparation of ethyl 3-amino-2,5-dioxopyrrolidine-3-carboxylate:

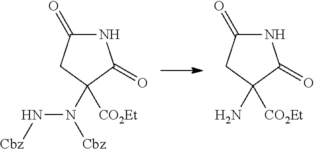

To a solution of ethyl 3-[N,N′-bis(benzyloxycarbonyl)hydrazino]-2,5-dioxopyrrolidine-3-carboxylate (496 mg) in acetic acid (15 ml) was added platinum oxide (102 mg). This mixture was stirred vigorously at 50° C. under hydrogen (atmospheric pressure) for 6 hours. During this reaction, to remove carbon dioxide generated with the progress of the reaction, the gas in the reactor was replaced with hydrogen gas several times. The reaction mixture was filtered through a Celite pad and then the Celite was washed with a small amount of acetic acid. The filtrate combined with the washers was concentrated and to the resulting residue was added toluene to remove azeotropically the residual acetic acid and then the mixture was concentrated again. To the residue was added ethyl acetate and the insoluble material was filtered off, and then the ethyl acetate solution was concentrated to give a crude product, which was then purified by a flash column chromatography (chloroform: methanol=30:1) to give the desired product (126 mg, 64%) as crystal. ¹H NMR (CDCl₃) data of this product were consistent with those of an optical active substance described in the above non-patent document. ¹H NMR (400 MHz, CDCl₃, 22° C.) δ: 4.28 (2H, q, J=7.1 Hz), 3.18 (1H, d, J=18.0 Hz), 2.76 (1H, d, J=18.0 Hz), 1.29 (3H, t, J=7.2 Hz).

### Example 10

Preparation of ethyl 3-amino-2,5-dioxopyrrolidine-3-carboxylate:

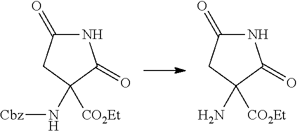

To a solution of ethyl 3-benzyloxycarbonylamino-2,5-dioxopyrrolidine-3-carbonate (1.00 g) in ethyl acetate (50 mL) was added 20% palladium hydroxide-carbon (0.50 g) and this mixture was stirred vigorously at room temperature under hydrogen flow (atmospheric pressure) for 1.5 hours. This reaction mixture was filtered through a Celite pad and the filtrate was concentrated to give the desired product (0.58 g, 100%) as white crystal.

### Example 11

Preparation of (S)-(+)-camphorsulfonic acid salt of ethyl (R)-3-amino-2,5-dioxopyrrolidine-3-carboxylate:

Ethyl 3-amino-2,5-dioxopyrrolidine-3-carboxylate (8.00 g) and (S)-(+)-camphorsulfonic acid (10.0 g) were dissolved in ethanol (80 ml) while warming, and this solution was concentrated under reduced pressure to about 45 ml in total. This solution was allowed to stand under ice-cooling and precipitated crystal was collected by filtering and washed with ethanol. This crystal was recrystallized from ethanol to give the desired product (4.70 g) as crystal.

Melting point: 229-230° C. (decomposition). [α]_{D}²⁷+10.2° (c 1.03, MeOH). ¹H NMR (400 MHz, D₂O, 23° C.) δ: 4.43 (2H, q, J=7.2 Hz), 3.56 (1H, d, J=18.8 Hz), 3.28 (1H, d, J=15.2 Hz), 3.22 (1H, d, J=18.8 Hz), 2.86 (1H, d, J=14.8 Hz), 2.46-2.37 (1H, m), 2.16 (1H, t, J=4.8 Hz), 2.09-2.00 (1H, m), 1.84 (1H, d, J=18.8 Hz), 1.68-1.61 (1H, m), 1.49-1.42 (1H, m), 1.30 (3H, t, J=7.2 Hz), 1.04 (3H, s), 0.83 (3H, s).

### Example 12

Preparation of ethyl (R)-2,5-dioxo-3-(pyrrol-1-yllpyrrolidine-3-carboxylate:

[symbol]

(S)-(+)-Camphorsulfonic acid salt of ethyl (R)-3-amino-2,5-dioxopyrrolidine-3-carboxylate (418 mg) was dissolved in 25% aqueous acetic acid solution (4 ml). Thereto were added sodium acetate (82 mg) and 2,5-dimethoxytetrahydrofuran (0.143 ml) and the mixture was stirred at 70° C. for 1.5 hours. After allowed to cool, to this mixture was added ethyl acetate (20 ml) and then the mixture was washed with water, followed by saturated brine and dried over magnesium sulfate and filtered. The filtrate was concentrated to give oil. This was purified by a flash column chromatography (hexane: ethyl acetate=3:1) to give the desired product (230 mg, 97%) as oil. ¹H NMR (CDCl₃) data were consistent with those described in the above non-patent document. ¹H NMR (400 MHz, CDCl₃, 23° C.) δ: 9.05 (1H, br), 6.94 (2H, t, J=2.2 Hz), 6.26 (2H, t, J=2.2 Hz), 4.28 (2H, q, J=7.2 Hz), 3.59 (1H, d, J=17.6 Hz), 3.36 (1H, d, J=18.0 Hz), 1.26 (3H, t, J=7.2 Hz). ¹³C NMR (100 MHz, CDCl₃, 24° C.) δ: 172.7, 170.5, 166.8, 120.0, 110.1, 68.6, 63.9, 41.9, 13.8. MS (APCI): 237(M+H).

### Example 13

Preparation of (3R)-2′-(4-bromo-2-fluorobenzyl)spiro[pyrrolidine-3,4′(1′H)-pyrrolo[1,2-a]pyrazine]-1′,2,3′,5(2H′)-tetraone:

(1) To a solution of ethyl (R)-2,5-dioxo-3-(pyrrol-1-yl)pyrrolidine-3-carboxylate (767 mg) in ethyl acetate (10 ml) was added trichloroacetyl chloride (1.1 ml) and this solution was heated under reflux overnight. This reaction mixture was allowed to cool to room temperature, and thereto was added trichloroacetyl chloride (1.1 ml) and this mixture was heated under reflux for 3 hours. This reaction mixture was allowed to cool with water to room temperature and the residual trichloroacetyl chloride was decomposed carefully with saturated aqueous sodium bicarbonate solution. After the aqueous layer was confirmed to be alkali, this mixture was extracted with ethyl acetate (5 ml) three times and the combined extract was washed with water and saturated brine successively, dried over magnesium sulfate, filtered and then concentrated to give a crude product as oil. This was purified by a flash column chromatography (n-hexane: ethyl acetate=1:1) to give ethyl (R)-2,5-dioxo-3-(2-trichloroacetylpyrrol-1-yl)pyrrolidine-3-carboxylate (1.17 g, 94%).

¹H NMR (400 MHz, DMSO-d₆, 22° C.) δ: 12.4 (br s, 1H), 7.68 (dd, 1H, J=1.2, 4.4 Hz), 7.55 (dd, 1H, J =1.6, 2.8 Hz), 6.44 (dd, 1H, J=2.4, 4.4 Hz), 4.25-4.08 (m, 2H), 3.72 (d, 1H, J=18.0 Hz), 3.06 (d, 1H, J=18.0 Hz), 1.11 (t, 3H, 7.2 Hz).

(2) To a solution of 4-bromo-2-fluorobenzylamine (0.93 g) and triethylamine (1.3 ml) in N,N-dimethylformamide (5 ml) was added a solution of ethyl

(R)-2,5-dioxo-3-(2-trichloroacetylpyrrol-1-yl)pyrrolidine-3-carboxylate (1.16 g) in N,N-dimethylformamide (3 ml) dropwise at room temperature. This mixture was stirred at room temperature for 8 hours. This reaction mixture was diluted with ethyl acetate, then washed with 1 M hydrochloric acid (three times), water (four times), and saturated brine successively, dried over magnesium sulfate, filtered and concentrated to give a crude product as yellow oil. This was purified by flash column chromatography (n-hexane: ethyl acetate =2:1) to give

(3R)-2′-(4-bromo-2-fluorobenzyl)spiro[pyrrolidine-3,4′(1′H)-pyrrolo[1,2-a]pyrazine]-1′,2,3′,5(2H′)-tetraone (831 mg, 65%). This product was further crystallized from n-hexane-ethyl acetate to give the desired product (385 mg) as crystal.

Mp: 189-191° C. ¹H NMR (400 MHz, DMSO-d₆, 22° C.) δ: 12.2 (br s, 1H), 7.73 (dd, 1H, J=2.0, 3.2 Hz), 7.55 (dd, 1H, J=2.0, 9.6 Hz), 7.36 (dd, 1H, J=2.0, 8.4 Hz), 7.17-7.12 (m, 2H), 6.53 (dd, 1H, J =2.8, 4.0 Hz), 5.04 (d, 1H, J=15.2 Hz), 4.96 (d, 1H, J=15.6 Hz), 3.57 (s, 2H).

### Comparative Example 1

Ethyl

3-benzyloxycarbonylamino-3-ethoxycarbonyl-3-cyanopropionate (2.0 g), Al₂O3 (1.5 g) and ethanol (5 mL) were mixed and the mixture was stirred for 8 hours while heating under reflux. However the progress of the reaction cannot be observed.

### Comparative Example 2

Ethyl

3-benzyloxycarbonylamino-3-ethoxycarbonyl-3-cyanopropionate (2.0 g), zinc chloride (78 mg), acetone oxime (168 mg), water (0.2 mL) and cumene (5 mL) were mixed and the mixture was stirred for 24 hours while heating under reflux. As the result of HPLC analysis on the reaction mixture, 7% of ethyl 2-benzyloxycarbonylamino-2-cyanoacetate, 6% of ethyl 3-benzyloxycarbonylamino-3-carbamoyl-3-ethoxycarbonylpropionate and 48% of ethyl 3-benzyloxycarbonylamino-2,5-dioxopyrrolidine-3-carboxylate were detected.

### Comparative Example 3

Ethyl

3-benzyloxycarbonylamino-3-ethoxycarbonyl-3-cyanopropionate (2.0 g), manganese dioxide (1.0 g), water (2.5 mL) and ethanol (5 mL) were mixed and the mixture was stirred for 1 hour while heating under reflux. The reaction solution was filtered through a Celite pad and then concentrated and the residue was purified by column chromatography on silica gel (n-hexane: ethyl acetate=100:0 to 0:100) to give the mixture (1.2 g) containing approximate equivalents of ethyl 3-benzyloxycarbonylamino-3-ethoxycarbonyl-3-cyanopropionate and ethyl 3-benzyloxycarbonylamino-3-cyanopropionate.

### INDUSTRIAL APPLICABILITY

The process of the present invention can prepare a 2,5-dioxopyrrolidine-3-carboxylates of the formula (I) in safe manner and efficiently. Among them, the compound of the formula (I) wherein R¹ is a hydrazino group protected with a protecting group can be derivatized to the compound wherein R¹ is converted into an amino group as shown in the Example 9. The patent documents 1 and 2 and the non-patent document 1 as aforementioned describe that this compound wherein R¹ is converted into an amino group and the compound of the formula (I) wherein R¹ is an amino group protected with a protecting group or pyrrol-1-yl group are applicable as an intermediate for Ranirestat, etc. Therefore the process of the present invention is useful as a process for preparing Ranirestat being useful as a therapeutic agent for diabetic neuropathic disorder showing a potent aldose reductase inhibitory activity, related compounds and intermediates thereof.

1. A process for preparing a compound of the following formula (I): 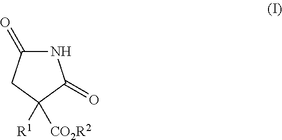
wherein R¹ is an amino group protected with a protecting group, a hydrazino group protected with a protecting group or a pyrrol-1-yl group; and R² is a lower alkyl group, a cycloalkyl group, a cycloalkyl-lower alkyl group, an optionally substituted aryl group, or an optionally substituted aryl-lower alkyl group,
which comprises the following steps (1) and (2):
(1) a step of converting a cyano group in a compound of the following formula (II): 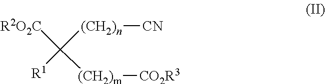
wherein n and m are each independently 0 or 1;
provided when n is 0 and m is 1, then R² and R³ are the same or different protecting groups for a carboxyl group; and when n is 1 and m is 0, then R² and R³ are the same protecting groups for a carboxyl group; and
R¹ is as defined above,
into a carbamoyl group in the presence of divalent palladium compound(s), primary amide(s) and water; and
(2) a step of cyclizing the product obtained in the step (1).

2. The process according to claim 1, wherein the divalent palladium compound is palladium(II) chloride, palladium(II) acetate or palladium(II) trifluoroacetate and the primary amide is acetamide, propionamide, n-butylamide or isobutylamide.

3. The process according to claim 1, wherein the step (1) is a step of converting a cyano group in the compound of the formula (II) into a carbamoyl group in the presence of divalent palladium compound(s), primary amide(s), water and organic solvent(s) without a cyano group.

4. The process according to claim 3, wherein the divalent palladium compound is palladium(II) chloride, palladium(II) acetate or palladium(II) trifluoroacetate, the primary amide is acetamide, propionamide, n-butylamide or isobutylamide, and the organic solvent without a cyano group is a single solvent selected from the group consisting of tetrahydrofuran, methanol, ethanol, isopropanol, tert-butanol, ethyl acetate, N,N-dimethylformamide and dimethyl sulfoxide or a mixed solvent of a combination of two or three kinds thereof.

5. The process according to claim 1, wherein the step (2) is a step of cyclizing the product obtained in the step (1) with base(s).

6. The process according to claim 5, wherein the step (2) is performed in the presence of chelating reagent(s).

7. The process according to claim 1, wherein the step (1) comprises a step of removing the divalent palladium compound(s) from the resulting reaction mixture of the step (1).

8. The process according to claim 7, wherein the step of removing the divalent palladium compound(s) from the resulting reaction mixture of the step (1) is a step of washing the resulting reaction mixture of the step (1) with aqueous inorganic acid solution(s).

9. The process according to claim 1 for preparing the compound of the formula (I) wherein R¹ is an amino group protected with a protecting group cleavable by hydrogenolysis, a hydrazino group protected with a protecting group cleavable by hydrogenolysis or a pyrrol-1-yl group, and R² is a lower alkyl group,
wherein the protecting group for a carboxyl group in the compound of the formula (II) is a lower alkyl group.

10. A process for preparing Ranirestat comprising a step of preparing the compound of the formula (I) from the compound of the formula (II) according to claim 1, and a step of converting the compound of the formula (I) into Ranirestat.
